(19) United States
(12) Patent Application Publication
(10) Pub. No.: US 2025/0002943 A1
(43) Pub. Date: Jan. 2, 2025

(54) METHODS FOR GENE EDITING

(71) Applicant: Genome Research Limited, Hinxton, Saffron Walden (GB)

(72) Inventors: Andrew BASSETT, Hinxton, Saffron Walden (GB); Valentina MIGLIORI, Hinxton, Saffron Walden (GB); Thomas BURGOLD, Hinxton, Saffron Walden (GB)

(21) Appl. No.: 18/695,454

(22) PCT Filed: Sep. 29, 2022
(86) PCT No.: PCT/GB2022/052473

§ 371 Date: Mar. 26, 2024

(30) Foreign Application Priority Data
Sep. 29, 2021 (GB) 2113933.2

(51) Int. Cl. C12N 15/90; C12N 9/22; C12N 15/11

(52) U.S. Cl. CPC C12N 15/907; C12N 9/22; C12N 15/11; C12N 2310/20; C12N 2800/80

(57) ABSTRACT

A single step method for gene editing using single-stranded oligo DNA nucleotide (ssODN) homology-directed repair (HDR) and a DNA recombinase is described. Systems, compositions and kits for one step gene editing are also described.

### FIELD OF INVENTION

The present invention relates to a one step method of gene editing. The invention also relates to systems, compositions and kits for one step gene editing.

### INTRODUCTION

The ability to engineer the genome of human cells is extremely important in the context of fundamental research, disease modelling and in the development of cellular therapeutics. For instance, the ability to tag endogenous genes with fluorescence markers, degrons or purification tags provides simple tracing of the dynamics, subcellular locations, and expression of genes, look at the effects of acute loss of protein products, and a means to purify protein complexes. Similarly, an ability to integrate large transgenes at specific sites is critical for reliable and regulated expression of transgenes in a therapeutic context. For examples, the use of “safe harbour” loci can ensure stable, high level expression of transgenes, or knock-ins at an endogenous gene can ensure appropriate expression and regulation. This has been shown to be particularly beneficial when targeting chimeric antigen receptors at the T cell receptor locus that can minimise T cell exhaustion, it may also be important in other contexts to maintain endogenous gene regulatory elements and genomic context.

CRISPR/Cas9 is a powerful tool that has been used to generate defined mutations, tag endogenous proteins and integrate transgenes at specific sites in the genome. However, especially for introduction of large exogenous sequences, these methodologies are limited in a number of ways. Firstly, it is often necessary to generate large homology directed repair (HDR) constructs for each specific integration site, which are time consuming to assemble. Secondly, DNA repair in most cells is biased against precise homology-directed repair, often resulting in poor efficiency and highly complex repair outcomes that are undesirable. Finally, the efficiency of the HDR process drops dramatically as the length of the inserted sequence increases, making it difficult to integrate large transgenes. As such there is a need to develop methods for the introduction of large exogenous sequences

### SUMMARY OF THE INVENTION

To circumvent some the limitations of the current methods for introducing large exogenous sequences, the present inventors have developed a technique that allows simple and efficient integration of large transgenes at essentially any site within the genome using a combination of CRISPR/Cas9 and site-specific recombination. The present method uses Cas endonuclease ribonucleoprotein complexes and short single stranded oligodeoxynucleotides as HDR templates to introduce a recombinase target site into a specific genomic locus. Simultaneously, a plasmid expressing a recombinase, e.g. the Bxb1 serine recombinase, and a universal cargo plasmid is used to integrate the cargo at this defined locus. This system avoids the need for cloning since all the components are common or chemically synthesised, and the use of a site-specific recombinase reduces the current limitation on the size of the cargo and results in a more homogeneous and precise editing event with fewer undesired mutations.

The benefits of the present gene editing method reside in the fact that precise, endogenous genomic integration can be achieved in just one step, as all the necessary reagents for the tagging are delivered into the cells with a single step, e.g. single electroporation (FIG. 1). It also allows the integration of large transgenes that would otherwise be difficult to integrate by direct CRISPR-mediated HDR.

In one embodiment the present invention relates to a method for gene editing comprising or consisting of
delivering simultaneously to a cell;
an endonuclease;
a targeting domain;
a single-stranded oligo DNA nucleotide (ssODN) homology-directed repair (HDR) template comprising a nucleotide sequence encoding a first recombination site;
a DNA recombinase; and
a cargo vector comprising a nucleotide sequence encoding a second recombination site and a cargo molecule.

In an embodiment the present invention relates to a system for gene editing comprising a Cas9 endonuclease, a single guide RNA (sgRNA), a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule selected from a fluorescence marker, a purification tag, a destabilising/degradation tag, or a chimeric antigen receptor (CAR), a selectable marker, a gene, a genomic region or cDNA for therapeutics, for differentiation, for metabolic engineering, a genetic locus, guide libraries for CRISPR screening, synthetic DNA fragments, an antibody or fragment thereof, a T-cell receptor, B-cell receptor formulated for simultaneous delivery to a cell.

In an embodiment the present invention relates to a kit comprising a Cas9 endonuclease, a single guide RNA (sgRNA), a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule selected from a fluorescence marker, a purification tag, a destabilising tag, or a chimeric antigen receptor (CAR), formulated for simultaneous delivery to a cell, and optionally instructions for use.

In an embodiment the present invention relates to a composition comprising or consisting of a Cas9 endonuclease, a single guide RNA (sgRNA), a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule selected from a fluorescence marker, a purification tag, a destabilising tag, or a chimeric antigen receptor (CAR), formulated for simultaneous delivery to a cell.

In an embodiment the present invention relates to a method for site specific integration of an exogenous polynucleotide sequence in a cell, comprising or consisting of
delivering to a cell a gene editing system, wherein the gene editing system comprises; an endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and an exogenous polynucleotide,
wherein the gene editing system is delivered to the cell simultaneously,
maintaining the cell under conditions such that the targeting domain directs endonuclease mediated integration of the first recombination site at a genomic site of interest,
maintaining the cell under conditions such that the DNA recombinase is expressed,
contacting the first recombination site and the second recombination site with the DNA recombinase, wherein, the DNA recombinase can mediate site-specific recombination between the first recombination site and the second recombination site to integrate the exogenous polynucleotide into the genomic site of interest.

In an embodiment the present invention relates to a method of producing a tagged protein comprising or consisting of
delivering simultaneously to a cell: an endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a tag,
maintaining the cell under conditions such that the targeting domain directs endonuclease mediated integration of the first recombination site at a genomic site that is in proximity to the nucleotide sequence encoding the protein that is to be tagged,
maintaining the cell under conditions such that the DNA recombinase is expressed,
allowing the DNA recombinase to contact the first recombination site and the second recombination site, wherein, the DNA recombinase can mediate site-specific recombination between the first recombination site and the second recombination site to integrate the tag into the genomic site of interest,
and optionally maintaining the cell under conditions such that expression of a tagged protein can occur.

In an embodiment the present invention relates to a cargo vector obtained by:
preparing a dsDNA comprising a nucleotide sequence encoding a cargo molecule and a recombination site, and
circularising the dsDNA to form the cargo vector.

### FIGURES

The invention is further described in the following non-limiting figures.

FIG. 1. Schematic of delivery of reagents for one step tagging. Five components are delivered into the cell simultaneously. Cas9 protein complexed with a guide RNA targets a double strand break at a specific genomic locus, and a single stranded DNA template is provided for homology directed repair to integrate an attP site. In addition, two plasmids are delivered encoding Bxb1 recombinase and a circularised cargo plasmid also containing an attB site.

FIG. 2. Temporally distinct targeting and recombination reactions. The genome editing and recombination reactions are temporally distinct. After delivery, the Cas9 ribonucleoprotein complex is immediately active to target the locus of interest and initiates homology directed repair with the ssDNA template, resulting in precise integration of the attP recombinase site into the genome (upper panel). Once the attP site is integrated it is no longer single stranded, so becomes a substrate for the Bxb1 integrase, that by this point is expressed from its plasmid. This triggers site specific recombination between the attP site in the genome and attB site on the cargo plasmid, resulting in integration of the cargo into the target site.

FIG. 3. Restriction digest to assess circularisation efficiency. The DraI restriction enzyme cuts once in the mNeonGreen cargo DNA, and can therefore be used to monitor circularisation efficiency. Linear PCR product (lane 2) when cut with DraI results in two bands (lane 3). After circularisation of the linear DNA by ligation, a number of bands are visible (lane 4). When this is digested with DraI (lane 5), a band corresponding to linearised circular DNA is predominant (asterisk), although there is still some non-circular product present as indicated by the two lower bands (arrowheads). Lane 1 and 6 are low and high molecular weight DNA size markers respectively.

FIG. 4. Tagging of ACTR10 with mNeonGreen in hiPSCs is efficient and precise. Panels show flow cytometry analysis of mNeonGreen fluorescence (x-axis) versus side scatter (y-axis) to determine integration efficiency of mNeonGreen into the ACTR10 locus. Upper panels show controls without Bxb1 and cargo plasmids (left), the single stranded HDR template (middle) or without any components, and show a low level of background integration of mNeonGreen (<0.38%). Lower panels show that when all components are delivered together, there is a large increase in the number of fluorescent tagged cells, up to 11.32%. The different panels show the effect of altering the amounts of Bxb1 plasmid (250 ng or 500 ng) or donor plasmid (96 ng, 192 ng or 384 ng). The optimal conditions are with the highest concentration of donor and Bxb1 plasmid (right panel).

FIG. 5. PCR analysis to detect targeted allele. This gel shows the results of PCR amplification across the site of integration in the conditions shown. In the absence of any editing (lane 7), or when no ssODN HDR template was added (lane 6), only the wt band is visible (wt). Upon addition of the ssODN (lanes 1 and ctrl), an additional band corresponding to the integration of the attP site is visible as would be expected (attP). Upon addition of Bxb1 and cargo plasmids (lanes 2-5), an additional upper band is visible corresponding to the correct integration of the mNeonGreen tag (tag). Consistent with the flow cytometry results, the highest efficiency is seen with the most Bxb1 and cargo plasmid (lane 5). Lane M contains DNA size markers.

### DETAILED DESCRIPTION OF THE INVENTION

The aspects and embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery.

An aspect of the present invention relates to a method for gene editing comprising or consisting of delivering simultaneously to a cell
an endonuclease;
a targeting domain;
a single-stranded oligo DNA nucleotide (ssODN) homology-directed repair (HDR) template or a prime editor guide RNA (pegRNA) template comprising a nucleotide sequence encoding a first recombination site;
a DNA recombinase; and
a cargo vector comprising a nucleotide sequence encoding a second recombination site and a cargo molecule.

In a preferred embodiment, the method includes delivery of a single-stranded oligo DNA nucleotide (ssODN) homology-directed repair (HDR) template and a prime editor guide RNA (pegRNA) template is not used.

The method of the invention utilises endonuclease driven integration of a first recombination site at a genomic site of interest. The endonuclease is directed to the genomic site of interest via a targeting domain. The targeting domain is designed to recognize a target DNA region of interest and directs the endonuclease to the target DNA region for editing. The endonuclease catalyses the formation of a double stranded break at the genomic site of interest. At the double stranded break homology-directed repair occurs, integrating the ssODN HDR template comprising a nucleotide sequence encoding the first recombination site. Once the first recombination site is integrated at the genomic site of interested it is recognised by the DNA recombinase. The DNA recombinase also recognises the second recombination site present in the cargo plasmid. Upon recognition of both recombination sites the DNA recombinase catalyses a unidirectional site-specific recombination of the cargo molecule at the genomic site of interest.

As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. These definitions generally refer to a single-stranded molecule, but in specific embodiments will also encompass an additional strand that is partially, substantially or fully complementary to the single-stranded molecule. Thus, a nucleic acid may encompass a single-stranded molecule or a double-stranded molecule that comprises one or more complementary strand(s) or “complement(s)” of a particular sequence comprising a molecule. As used herein, a single-stranded nucleic acid may be denoted by the prefix “ss”, a double stranded nucleic acid by the prefix “ds”, and a triple stranded nucleic acid by the prefix “ts”. The term “gene” may refer to the segment of DNA involved in producing a polypeptide chain, it includes regions preceding and following the coding region as well as intervening sequences (introns and non-translated sequences, e.g., 5′- and 3′-untranslated sequences and regulatory sequences) between individual coding segments (exons). A gene can also be an amplified nucleic acid molecule produced in vitro comprising all or a part of the coding region and/or 5′- or 3′-untranslated sequences linked thereto.

Targeted genome modification or targeted genome editing is known in the art as a genome engineering technique that uses targeted DNA double-strand breaks (DSBs) to stimulate genome editing through homologous recombination (HR)-mediated recombination events or other repair mechanisms. To achieve effective genome editing via introduction of site-specific DNA DSBs, four major classes of customizable DNA binding proteins have been used in the art: meganucleases derived from microbial mobile genetic elements, ZF nucleases based on eukaryotic transcription factors, rare-cutting endonucleases/sequence specific endonucleases (SSN), for example TALENs, transcription activator-like effectors (TALENs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALEN proteins all recognize specific DNA sequences through protein-DNA interactions. Although meganucleases integrate its nuclease and DNA-binding domains, ZF and TALEN proteins consist of individual modules targeting 3 or 1 nucleotides (nt) of DNA, respectively. ZFs and TALENs can be assembled in desired combinations and attached to the nuclease domain of FokI to direct nucleolytic activity toward specific genomic loci. Zinc finger proteins have specific DNA binding properties and can be targeted to specific DNA sequences via the zinc finger binding domain. The present invention may use zinc finger nucleases or TALENs to catalyse the integration of the first recombination site.

Upon delivery into host cells via the bacterial type III secretion system, TAL effectors enter the nucleus, bind to effector-specific sequences in host gene promoters and activate transcription. Their targeting specificity is determined by a TAL effector DNA binding domain, a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471 (all incorporated herein by reference). Customized plasmids can be used with the Golden Gate cloning method to assemble multiple DNA fragments. The Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct.

Another genome editing method is CRISPR. The use of this technology in genome editing is well described in the art, for example in U.S. Pat. No. 8,697,359, incorporated herein by reference. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage. Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer).

The Type II CRISPR is one of the most well characterized systems and carries out targeted DNA double-strand breaks in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the type II CRISPR-Cas system, and a large monomeric DNA nuclease guided to a DNA target sequence adjacent to the PAM sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with a guide RNA (gRNA) also called single guide RNA (sgRNA) can introduce site-specific double strand breaks (DSBs) into genomic DNA of live cells from various organisms. For applications in eukaryotic organisms, codon optimized versions of Cas9, which is originally from the bacterium Streptococcus pyogenes, have been used. It is also possible to use alternative nucleases to Cas 9 for example Cpf1 (also known as Cas12a), MAD7 (an engineered nuclease of the Class 2 type V-A CRISPR-Cas (Cas12a/Cpf1) family) and related nucleases.

In an embodiment of the present method a synthetic CRISPR system is used comprises two components the gRNA (also known as sgRNA) and the Cas endonuclease. The sgRNA is a specific RNA sequence that recognizes the target DNA region of interest and directs the Cas nuclease there for editing. The gRNA is made up of two parts: crispr RNA (crRNA), a 17-20 nucleotide sequence complementary to the target DNA, and a tracr RNA, which serves as a binding scaffold for the Cas nuclease. A ribonucleoprotein (RNP) complex can be used to deliver the nuclease in complex with the gRNA into the target cell. Using an RNP for delivery has a number of advantages including that is does not leave exogenous sequences in the genome thereby reducing potential for off target effects, it can also be used in cells which are difficult to transfect. Furthermore, in the present invention where a RNP is used to deliver the Cas9 to the cell, this means that the Cas9 protein will be immediately active and can direct the targeted integration of the attP site at the genomic site of interest. The gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA. The genomic target can be any approximately a 20 nucleotide DNA sequence, provided that the target is present immediately upstream of a PAM sequence.

In an embodiment the endonuclease is a DNA endonuclease. In an embodiment the endonuclease is selected from a Cas9 endonuclease, a Cpf1 (Cas12a) endonuclease, CasX endonuclease, CasY endonuclease, MAD7 endonuclease, TALEN, zinc finger nuclease, prime editor, genetically modified Cas endonuclease.

The endonuclease can be targeted to specific regions of the genome via targeting domains. The targeting domain may be part of the nuclease of conjugated to the nuclease or may be a separate moiety from the nuclease. For example, where a Cas endonuclease is used in the method of the invention the targeting domain is a single guide RNA. Where a zinc finger nuclease is used in the method of the invention, the targeting domain is a zinc-finger DNA binding domain. The zinc finger DNA binding domain can be assembled in desired combinations to target various sequences. Where a transcription activator-like effector nuclease (TALEN) is used in the method of the invention the targeting domain is a transcription activator-like effector DNA binding domain. Transcription activator-like effector DNA binding domain comprises of tandem repeats of about 34 amino acids per repeat. Each repeat contains two variable di-residues (RVD) at amino acid position 12 and 13 that confer DNA base recognition. In an embodiment the targeting domain may be selected from an sgRNA), a zinc finger DNA binding domain, transcription activator-like effector DNA binding domain.

Where Cas9 is used in the method of the invention, once expressed, the Cas9 protein and the gRNA form a ribonucleoprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a composed of more than one discrete polynucleotides. The guide polynucleotide sequence can be an RNA sequence, a DNA sequence, or a combination thereof (a RNA-DNA combination sequence). Used herein the guide polynucleotide is an RNA sequence referred to as a guide RNA (sgRNA). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also The terms “target site”, “target sequence”, “target DNA”, “target locus”, “genomic target site”, “genomic target sequence”, and “genomic target locus” are used interchangeably herein and refer to a polynucleotide sequence in the genome (including chloroplastic and mitochondrial DNA) of a cell at which a double-strand break is induced in the cell by a Cas endonuclease. The target site can be a regulatory region that influences expression of the target regulatory sequence.

In an embodiment the guide nucleic acid may be designed using suitable algorithms such as Dharmacon™ Edit-R™, crOATAN, JACKS, Vienna bioscore, KS score which can be used to design highly potent guide RNAs. The gRNAs may be provided in a vector which may be designed for a single gRNA.

As used herein the term “single-stranded oligo DNA nucleotide (ssODN) homology-directed repair (HDR) template” refers to a single stranded donor oligonucleotide which is used to insert or change a short nucleotide sequence at an endogenous genomic target region. These donor nucleotides are used as part of the homology directed repair mechanism. Homology-directed repair (HDR) is a process of homologous recombination where a DNA template is used to provide the homology necessary for precise repair of a double-strand break (DSB). As such in the present method when a double stranded break is introduced at the genomic site of interest catalysed by the Cas endonuclease, the ssODN HDR template is used to repair the double stranded break, via a homology directed repair mechanism. The ssODN HDR template comprises a nucleotide sequence encoding the first recombination site, as such the first recombination site is inserted into the genomic site of interest via homology directed repair.

In an embodiment of the present invention a prime editor guide RNA (pegRNA) may be used as an alternative option to provide the nucleotide sequence encoding a first recombination site. A pegRNA is an sgRNA with a primer binding sequence and the template containing the desired RNA sequence added at the 3′ end. As such, where a pegRNA is used, the targeting domain and the pegRNA may be part of the same molecule.

As used herein the terms “first recombination site” and “second recombination site” refer specific recombination sites. These sites direct site-specific recombination wherein DNA strand exchange occurs between nucleotide sequences have a certain degree of sequence homology. Site-specific recombination involves two short DNA sequences (recombination sites) which may be within the same molecule or in different molecules. A recombinase enzyme recognizes the recombination sites and then promotes a rearrangement of the DNA. This rearrangement requires recombinase-catalyzed breaking and rejoining of both DNA strands in each site. Depending on the DNA recombinase that is to be used, different first and second recombination sites may be required, the selection of suitable recombination sites is within the capabilities of the skilled person.

In the present methods, once the first recombination site is integrated within the genome, and therefore provides a double stranded sequence, the DNA recombinase can recognise this first recombination site and the second recombination site encoded within the cargo vector. Upon recognition of both sites the DNA recombinase can mediate site-specific recombination between the first recombination site and the second recombination site which results in the introduction of the nucleotide sequence encoding the cargo molecule into the genomic site of interest.

The inventors remarkably found that the methods of the invention can advantageously be carried out in a single step method with simultaneous delivery of the components. The inventors have surprisingly found that the recombinase does not act on the ssODN HDR template and only acts on the double stranded DNA once the ssODN HDR template is used to repair the double stranded break, via a homology directed repair mechanism.

In an embodiment the method for gene editing, comprises
delivering simultaneously to a cell; a Cas endonuclease, a single guide RNA (sgRNA), a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule.

Once the Cas endonuclease, the single guide RNA (sgRNA), a single-stranded oligo DNA nucleotide (ssODN) homology-directed repair (HDR) template comprising a nucleotide sequence encoding a first recombination site, the DNA recombinase and the cargo vector comprising a nucleotide sequence encoding a second recombination site and a cargo molecule, i.e. the gene editing components, are delivered to the cell these components then direct the editing of the cell genome. Once within the cell the sgRNA guides the Cas endonuclease to the genomic site of interest. The Cas endonuclease catalyses the formation of a double stranded break which is repaired via HDR using the ssODN HDR template resulting in the integration of the first recombination site at the genomic site of interest.

Once the first recombination site is integrated into the genomic site of interest and becomes dsDNA, it is recognised by the DNA recombinase. The second recombination site present in the cargo vector is also recognised by the DNA recombinase, the DNA recombinase can mediate site-specific recombination between the first recombination site and the second recombination site to integrate the nucleotide sequence encoding the cargo molecule into the genomic site of interest when both first and second recombination sites are recognised by the DNA recombinase.

The DNA recombinase may be provided to the cell in a number of different formats. For example the DNA recombinase may be provided as; a plasmid or vector comprising a nucleotide sequence encoding the DNA recombinase, an mRNA encoding the DNA recombinase or as a polypeptide.

Preferably, the DNA recombinase is provided/delivered in a plasmid or vector, e.g. an expression vector that comprises a nucleotide sequence encoding the DNA recombinase. The DNA recombinase is therefore expressed in the cell once the components have been delivered and it is not delivered as a protein. Delivery in an expression vector delays expression of the recombinase protein. This allows the other components, preferably delivered as a protein and/or RNA to act first, i.e. it allows the endonuclease to act first to include the double stranded break which is then repaired via HDR using the ssODN HDR template. In other words, this allows double stranded breakage and integration of the ssODN HDR template with the recombinase site to occur first. Once it is expressed, the recombinase acts on the double stranded DNA which now includes a recombination site. This also avoids recombination of the cargo molecules with the donor template prior to integration and double stranded DNA formation.

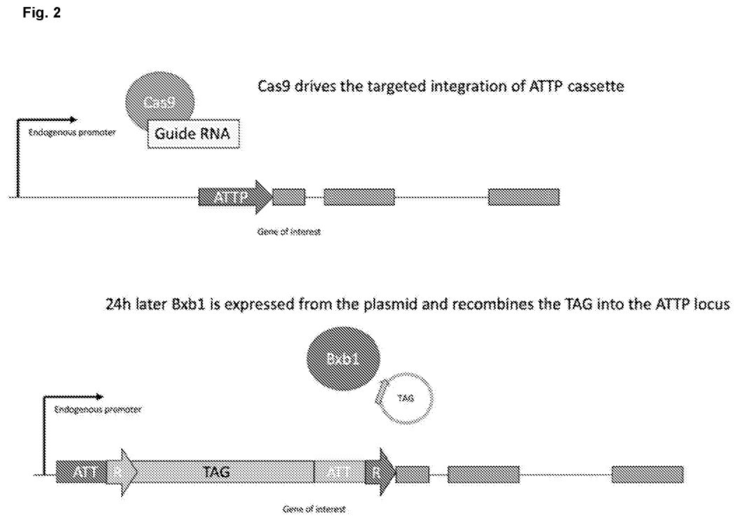

Expression of the recombinase protein may occur after at least 12 hours, e.g. after about 24 hours to about 48 hours. Expression may peak around 48 h after delivery to the cell. This is also illustrated in FIG. 2.

The expression vector may include additional sequences that would be known to a skilled person, including promoter sequences. Such promoter sequences can be further used to fine tune timing of the expression of the recombinase, for example by using an inducible promoter.

Where the DNA recombinase is provided as a vector or an mRNA which encodes the DNA recombinase, the cell will then express the DNA recombinase. The DNA recombinase is capable of recognising a double stranded DNA substrate as such the DNA recombinase will only recognise the first recombination site once it is integrated at the genomic site of interest.

The endonuclease is preferably provided as a protein or ribonucleoprotein complex in the case of RNA-guided endonucleases.

As such once the gene editing components are delivered to the cell the method may optionally include the further steps of:
maintaining the cell under conditions such that the targeting domain directs endonuclease mediated integration of the first recombination site at a genomic site of interest,
maintaining the cell under conditions such that the DNA recombinase is expressed,
allowing the DNA recombinase to recognise the integrated first recombination site and the second recombination site encoded by the cargo vector, wherein, the DNA recombinase can mediate site-specific recombination between the first recombination site and the second recombination site to integrate the nucleotide sequence encoding the cargo molecule into the genomic site of interest.

In certain embodiments the method for gene editing does not comprise any further steps which comprise delivering components to the cell. As mentioned above the delivery of the gene editing components is performed simultaneously in a one step manner.

As used herein the term “simultaneously” means that the components are delivered to the cell at the same time. The present method does not deliver the gene editing components in separate steps. For example, if the gene editing components are delivered by nucleofection then a single nucleofection step is required to deliver the endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule, to the cell.

A key benefit of the present method is that all of the editing components are delivered to the cell in a single process which provides a streamlined and efficient gene editing method. As such the gene editing components may be delivered by any suitable method that allows simultaneous delivery of an endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule, to a cell.

In an embodiment the gene editing components i.e. the endonuclease, the targeting domain, the single-stranded oligo DNA nucleotide (ssODN) homology-directed repair (HDR) template comprising a nucleotide sequence encoding a first recombination site, the DNA recombinase and the cargo vector comprising a nucleotide sequence encoding a second recombination site and a cargo molecule are delivered to the cell simultaneously via electroporation, nucleofection, transfection, or viral gene transfer. An advantage of the present method is that the gene editing components can be delivered in a simultaneous manner, as this results in a streamlined one step process for gene editing. In a preferred embodiment the gene editing components are delivered via nucleofection. Nucleofection is an electroporation-based transfection method which enables transfer of nucleic acids such as DNA and RNA into cells by applying a specific voltage and reagents. Nucleofection results in delivery of DNA and RNA directly to the nucleus and therefore allows for efficient transfection of non-dividing cells as well as dividing cells.

The Cas endonuclease may be selected from any Cas endonuclease which can catalyse the formation of a double stranded break. Suitable Cas endonucleases include Cas9 endonuclease, Cpf1 (Cas12a) endonuclease, CasX endonuclease, CasY endonuclease, MAD7 endonuclease. Natural variants of Cas endonuclease from other species may be suitable. Engineered and modified versions of Cas endonucleases may be suitable for example Cas endonucleases with different PAM specificities, different on/off target activities or nickase versions. MAD7 may also be suitable which is an engineered class 2 type V-A CRISPR-Cas (Cas12a/Cpf1) system.

In an embodiment the Cas endonuclease is delivered as a ribonucleoprotein (RNP) complex. As mentioned above, providing the Cas endonuclease as an RNP complex has a number of advantages as it does not leave exogenous sequences in the genome thereby reducing potential for off target effects, it can also be used in cells which are difficult to transfect. Furthermore, in the present invention where a RNP is used to deliver the Cas9 to the cell, this means that the Cas9 protein will be immediately active and can immediately direct the targeted integration of the attP site at the genomic site of interest.

DNA recombinases are enzymes generally derived from bacteria or fungi which can catalyse the directionally sensitive DNA exchange reactions between short target site sequences that are specific to each recombinase. The target sequence may be between 25-60 nucleotides in length. In an embodiment the DNA recombinase is a serine recombinase, for example Bxb1 or phiC31. In an embodiment the DNA recombinase is selected from; Bxb1, phiC31, Cre, Flp, KD, B2, B3, or R. Flp (also known as flippase) recombinase is derived from Saccharomyces cerevisiae and recognizes a pair of FLP recombinase target (FRT) sequences. Cre recombinase is encoded by the P1 bacteriophage cyclization recombination gene and recognizes pairs of LoxP sites. PhiC31 integrase is a serine recombinase derived from Streptomyces phage φC31, phiC31 catalyses site-specific recombination between its corresponding attP and attB recombination sites. Bxb1 is a large serine recombinase derived from mycobacteriophage, Bxb1 catalyses site-specific recombination between its corresponding attP and attB recombination sites. In a preferred embodiment the serine recombinase is Bxb1. The use of Bxb1 has advantages as it has been shown to be functional and non-toxic in mammalian cells, and is able to catalyse highly efficient unidirectional recombination between short heterologous attP and attB target sites.

In an embodiment the first recombination site is selected from attB, attP, attL, attR, LoxP, FRT. In an embodiment the second recombination site is selected from attB, attP, attL, attR, LoxP, FRT. As mentioned above a suitable first and second recombination site should be selected based on the specificity of the DNA recombinase that will be used. In a preferred embodiment the first recombination site is selected from attB or attP. In a preferred embodiment the second recombination site is selected from attB or attP. In an embodiment the DNA recombinase is Bxb1 and the first recombination site is attP and second recombination sites is attB. In an embodiment the DNA recombinase is Bxb1 and the first recombination site is attB and second recombination sites is attP. In an embodiment the DNA recombinase is phiC31 and the first recombination site is attP and second recombination sites is attB. In an embodiment the DNA recombinase is phiC31 and the first recombination site is attB and second recombination sites is attP.

The gene editing method of the invention can be used for a variety of applications including to engineer exogenous polypeptide or genetic information into a genome for example for therapeutic purposes. The present gene editing method allows large transgenes to be inserted into the genome as such the cargo vector may comprise a gene, multiple genes, genomic regions, cDNA, or a genetic locus. Genomic regions or cDNA for therapeutics, for differentiation, for metabolic engineering may be encoded within the cargo vector. For example, genomic regions or cDNA for therapeutics may include dystrophin, dopamine production enzymes. Genomic regions or cDNA for differentiation may include transcription factor combinations to drive production of specific cell types. Genomic regions or cDNA for metabolic engineering may include enzyme cascades. The present method may be used to incorporate libraries of coding and non-coding sequences for example for use in saturation genome editing. The present method may be used to edit the genome of an animal to produce a transgenic animal for example the humanisation of a mouse genome by incorporating a human genetic locus, this may be used in the production of human antibodies within a transgenic animal. The present method may be used to incorporate non-genic sequences and the cargo vector may encode, for example, guide libraries for CRISPR screening, synthetic DNA fragments. Furthermore, the cargo vector may encode a chimeric antigen receptor or an antibody or a fragment thereof. Other applications include to engineer tagged proteins for research purposes. For example, the tag may be a fluorescence marker, a purification tag, a degradation tag or a selectable marker.

As used in the various aspects, including the products and methods of the invention, herein, the cargo molecule may comprise a fluorescence marker, a purification tag, a destabilising/degradation tag, or a chimeric antigen receptor (CAR), a selectable marker, a gene, a genomic region or cDNA for therapeutics, for differentiation, for metabolic engineering, a genetic locus, guide libraries for CRISPR screening, synthetic DNA fragments, an antibody or fragment thereof, a T-cell receptor and/or a B-cell receptor.

In an embodiment the cargo vector encodes a cargo molecule selected from; a fluorescence marker, a purification tag, a degradation tag, or a chimeric antigen receptor (CAR). The degradation tag may be selected from an N-degron or a C-degron, preferably the degradation tag is selected from; AID, HALO or DHFR. The fluorescence marker may be selected from GFP, YFP, CFP, BFP, RFP, EGFP, mCherry, mStrawberry, mOrange, dTomato. The purification tag may be selected from Halo, SNAP, TAP, CLIP, FLAG, c-Myc, CBP, or hexa histidine. The selectable marker is selected from puromycin resistance gene, blasticidin resistance gene, neomycin resistance gene or hygromycin resistance gene.

As used herein the term “cargo vector” is used to refer to a vector which encodes the exogenous polynucleotide sequence which is to be integrated at the genomic site of interest. The exogenous polynucleotide may encode a cargo molecule as described herein. As such the cargo vector comprises a nucleotide sequence encoding a cargo molecule and components required for successful incorporation of the nucleotide sequence encoding the cargo molecule. In particular the cargo vector comprises a nucleotide sequence encoding a second recombination site, as this allows for site specific recombination between the second recombination site and the first recombination site that is integrated into the genome directed by the DNA recombinase. The cargo vector may be double stranded.

The methods according to the invention may comprises a step of preparing the cargo vector, comprising:
preparing a dsDNA comprising a nucleotide sequence encoding a cargo molecule and a second recombination site, and
circularising the dsDNA to form the cargo vector.

Circularisation of the dsDNA to form the cargo vector may comprise digesting the dsDNA with appropriate restriction enzymes and self-ligation. The dsDNA may be a PCR product. The skilled person will be aware of how to select the appropriate restriction enzymes. The step of preparing the cargo vector is performed prior to the step of delivering the gene editing components to the cell. By preparing the cargo vector by preparing a dsDNA comprising a nucleotide sequence encoding a cargo molecule and a second recombination site, and circularising the dsDNA to form the cargo vector, this results in a scarless circular vector which does not comprise extraneous plasmid sequences. In the method of the invention a single first recombination site, such as an attP site, may be used. As such the entirety of the cargo vector may be introduced at the genomic site of interest. By using a scarless cargo vector this reduces the possibility of introducing extraneous sequence at the genomic site of interest. Furthermore, by using a scarless cargo vector it allows for correct in frame insertion of the nucleotide sequence encoding the cargo molecule.

In an embodiment the cargo vector consists of a nucleotide sequence encoding a cargo and a second recombination site. As such, in an embodiment the cargo vector does not comprise any extraneous nucleotide sequence.

According to the methods of the invention more than one cargo vector may be delivered to the cell simultaneously. More than one homology-directed repair (HDR) template comprising a nucleotide sequence encoding first recombination site may also be delivered to the cell simultaneously. More than one HDR template may be used to introduce more than one first recombination site. The more than one first recombination sites may be the same or different. The more than one first recombination sites may direct integration of the cargo at the same of different genomic sites. As such, the method may comprise delivering more than one targeting domain to target the integration of the more than one first recombination sites. The more than one targeting domains may be designed to target the same or different genomic region. Where more than one HDR template is delivered to the cell these may be used to simultaneously integrate multiple cargo at distinct positions within the genome, alternatively they may be used to integrate multiple cargo at the same position.

In an embodiment the gene editing components may be delivered to the cell in vivo, in vitro or ex vivo. In some embodiments, the gene editing components are delivered to prokaryotic or eukaryotic cells. The cells may be a stem cell or progenitor cell. When stem cells are used in the practice of the invention, the stem cells may be multipotent adult stem cells or pluripotent embryonic stem cells. Wherein the cell is selected from a mammalian cell or a plant cell, preferably wherein the cell is selected from an iPSC or a stem cell.

The methods of the present invention may further comprise performing steps which bias cell repair mechanisms towards HDR and integration of the ssODN, these steps may include exposing the cell to DNA-PK inhibitors and or POLQ inhibitors. The method may also comprise a step of performing cold shock of the cells, which may help to bias cell repair mechanisms towards HDR and integration of the ssODN. The cold shock may be performed at approximately 28° C. to 35° C., preferably at 32° C.

An aspect of the present invention relates to a system for gene editing comprising a Cas9 endonuclease, a single guide RNA (sgRNA), a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule selected from a fluorescence marker, a purification tag, a destabilising tag, or a chimeric antigen receptor (CAR), formulated for simultaneous delivery to a cell.

In an embodiment the system for gene editing may comprise one or more cargo vector. The system may comprise one or more targeting domain. The system may comprise one or more SSODN.

An aspect of the invention relates to a kit comprising a Cas9 endonuclease, a single guide RNA (sgRNA), a single-stranded oligo DNA nucleotide (ssODN) homology-directed repair (HDR) template comprising a nucleotide sequence encoding a first recombination site, a vector comprising a nucleotide sequence encoding a DNA recombinase and a cargo vector comprising a nucleotide sequence encoding a second recombination site and a cargo molecule selected from a fluorescence marker, a purification tag, a destabilising tag, or a chimeric antigen receptor (CAR), formulated for simultaneous delivery to a cell, and optionally instructions for use.

The kit according to the invention may further comprise components which optimise simultaneous delivery of the Cas9 endonuclease, the single guide RNA (sgRNA), the single-stranded oligo DNA nucleotide (ssODN) homology-directed repair (HDR) template comprising a nucleotide sequence encoding a first recombination site, the DNA recombinase and the cargo vector comprising a nucleotide sequence encoding a second recombination site and a cargo molecule selected from a fluorescence marker, a purification tag, a destabilising tag, or a chimeric antigen receptor (CAR), i.e. the gene editing components. For example, the kit may comprise reagents which optimise simultaneous delivery of the gene editing components via electroporation, nucleofection, transfection, or viral gene transfer. These reagents may also be cell type specific reagents. In a preferred embodiment the kit comprises reagents which optimise simultaneous delivery of the gene editing components via nucleofection.

The kit may also further comprise components which bias cell repair mechanisms towards HDR and integration of the ssODN, these components may include DNA-PK inhibitors and or POLQ inhibitors. The kit may also comprise components for performing cold shock of the cells. The cold shock may be performed at approximately 28° C. to 35° C., preferably at 32° C.

An aspect of the present invention relates to a composition comprising a Cas9 endonuclease, a single guide RNA (sgRNA), a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a cargo molecule selected from a fluorescence marker, a purification tag, a destabilising tag, or a chimeric antigen receptor (CAR), formulated for simultaneous delivery to a cell.

The composition may comprise additional reagents to optimise simultaneous delivery of the Cas9 endonuclease, the single guide RNA (sgRNA), the single-stranded oligo DNA nucleotide (ssODN) homology-directed repair (HDR) template comprising a nucleotide sequence encoding a first recombination site, the DNA recombinase and the cargo vector comprising a nucleotide sequence encoding a second recombination site and a cargo molecule selected from a fluorescence marker, a purification tag, a destabilising tag, or a chimeric antigen receptor (CAR), i.e. the gene editing components.

An aspect of the present invention relates to a method for site specific integration of an exogenous polynucleotide sequence in a cell, comprising
delivering to a cell a gene editing system, wherein the gene editing system comprises; a endonuclease, a targeting domain a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and an exogenous polynucleotide, wherein the gene editing system is delivered to the cell simultaneously,
maintaining the cell under conditions such that the targeting domain directs endonuclease
mediated integration of the first recombination site at a genomic site of interest, optionally maintaining the cell under conditions such that the DNA recombinase is expressed,
contacting the first recombination site and the second recombination site with the DNA recombinase, wherein, the DNA recombinase can mediate site-specific recombination between the first recombination site and the second recombination site to integrate the exogenous polynucleotide into the genomic site of interest.

An aspect of the present invention relates to a method of producing a tagged protein comprising;
delivering simultaneously to a cell; an endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a tag,
maintaining the cell under conditions such that the targeting domain directs endonuclease mediated integration of the first recombination site at a genomic site that in proximity of the nucleotide sequence encoding the protein that is to be tagged,
optionally maintaining the cell under conditions such that the DNA recombinase is expressed,
allowing the DNA recombinase to contact the first recombination site and the second recombination site, wherein, the DNA recombinase can mediate site-specific recombination between the first recombination site and the second recombination site to integrate the tag into the genomic site of interest,
and optionally maintaining the cell under conditions such that expression of a tagged protein can occur.

In an embodiment the genomic site at which the nucleotide sequence encoding the tag is integrated is in proximity of the nucleotide sequence encoding the protein that is to be tagged, therefore the tag may be integrated upstream or downstream or internally within the nucleotide sequence. In an embodiment the genomic site at which the nucleotide sequence encoding the tag is integrated is upstream of the nucleotide sequence encoding the protein to be tagged. The term “upstream” refers to the 5′ end of the coding strand of DNA. For example, the nucleotide sequence encoding the tag may be integrated at the 5′ end of the open reading frame. In a preferred embodiment the nucleotide sequence encoding the tag may be integrated at the 5′ end of the open reading frame but downstream of the start codon. In an embodiment the genomic site at which the nucleotide sequence encoding the tag is integrated is downstream of the nucleotide sequence encoding the protein to be tagged. The term “downstream” refers to the 3′ end of the coding strand of DNA. For example, the nucleotide sequence encoding the tag may be integrated at the 3′ end of the open reading frame.

In an embodiment the method for producing a tagged protein may comprise two stages. The first stage may comprise engineering a cell to encode a tagged protein and the second stage may comprise expressing said tagged protein. The first stage may comprise the steps of
delivering simultaneously to a cell; an endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a tag,
maintaining the cell under conditions such that the targeting domain directs endonuclease mediated integration of the first recombination site at a genomic site that is in proximity of the nucleotide sequence encoding the protein that is to be tagged,
optionally maintaining the cell under conditions such that the DNA recombinase is expressed,
allowing the DNA recombinase to contact the first recombination site and the second recombination site, wherein, the DNA recombinase can mediate site-specific recombination between the first recombination site and the second recombination site to integrate the tag into the genomic site of interest,

The second stage may comprise the step of
maintaining the cell under conditions such that expression of a tagged protein can occur.

The step of maintaining the cell under conditions such that expression of a tagged protein can occur may comprise maintaining the cell under specific temperature, nutrient level and agitation such that the cells can multiply. Conditions for successful cell culture and protein expression are known within the art and the skilled person can determine suitable expression conditions using routine methods.

An aspect of the present invention relates to a cargo vector obtained by:
preparing a dsDNA comprising a nucleotide sequence encoding a cargo molecule and a recombination site, and
circularising the dsDNA to form the cargo vector.

A cargo vector obtained by preparing a dsDNA comprising a nucleotide sequence encoding a cargo molecule and a recombination site, and circularising the dsDNA to form the cargo vector, is a scarless vector i.e. it does not comprise extraneous plasmid sequence. A cargo vector produce by this method may be for use in the gene editing, site-specific integration, or tagged protein methods of the present invention.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further illustrated in the following non-limiting examples.

### EXAMPLES

### Example 1. Principle of One-Step Tagging Methodology

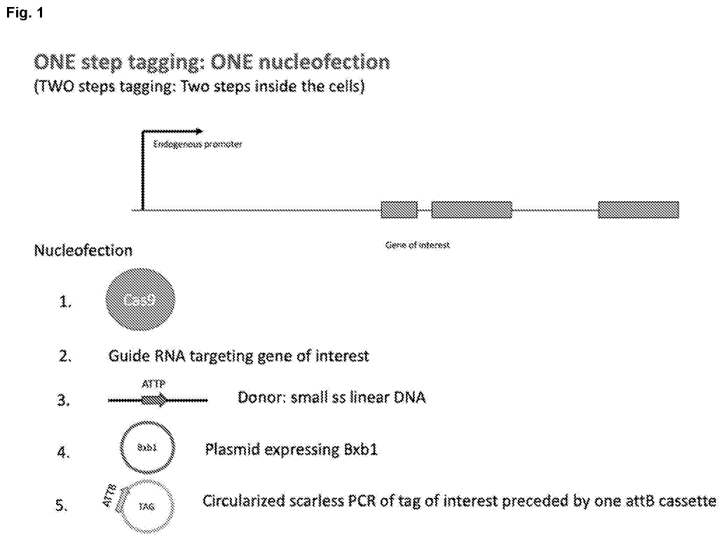

Five components are delivered simultaneously into cells by electroporation: recombinant Cas9 protein, a synthetic sgRNA, a chemically synthesised ssODN HDR template containing the attP site, a plasmid expressing the Bxb1 recombinase and a cargo plasmid (FIG. 1).

Since Cas9 is delivered as a ribonucleoprotein (RNP) complex, it will be immediately active, and direct the targeted integration of the attP cassette into the genomic site of interest. The Bxb1 plasmid will express the integrase protein after several hours, but since it requires a double stranded DNA substrate, it can only recognise the attP site after genomic integration. Once this has occurred, Bxb1 catalyses a unidirectional site-specific recombination of the chosen cargo into the CRISPR-targeted locus (FIG. 2).

An advantage of this method is that it is highly modular and does not require cloning of homology constructs. The choice of target site can be altered very simply by altering the sequence of the sgRNA and ssODN, which are ordered as chemically synthesised oligonucleotides from standard suppliers. The Bxb1 and cargo plasmids are universal to any target site, and it is possible to assemble a set of cargo plasmids for fluorescent, degron or purification tags, or other specific cargos (e.g. CARs). Since the entire cargo plasmid is integrated, we generate these by circularising a PCR product consisting of the cargo preceded by an attB site to avoid additional exogenous plasmid sequence. This is also advantageous since it reduces the chances of integration of plasmid-derived sequences, which is particularly important in therapeutic applications.

### Example 2. Fluorescent Tagging of ACTR10 Gene

To test and optimise the method, we chose to tag the highly expressed ACTR10 gene by direct fusion with a mNeonGreen fluorophore at the 5′ end of the open reading frame. We thus designed a sgRNA and a 200 nt ssODN HDR template to integrate the attP sequence just downstream of the start codon. The ssODN consisted of a 58 nt attP site flanked by 71 nt homology arms on either side.

Since we use a single attP site, the entirety of the circular cargo plasmid is integrated into the genome. To avoid insertion of extraneous sequence and to allow correct, in frame integration of the tag at the ACTR10 gene, we generated a circularised mNeonGreen cargo, preceded by an attB cassette. This was achieved by PCR amplification of the appropriate fragment with primers containing type IIS restriction sites (Bbs I) sites. Digestion and self-ligation of the PCR product generated a scarless, circular DNA without extraneous plasmid sequences.

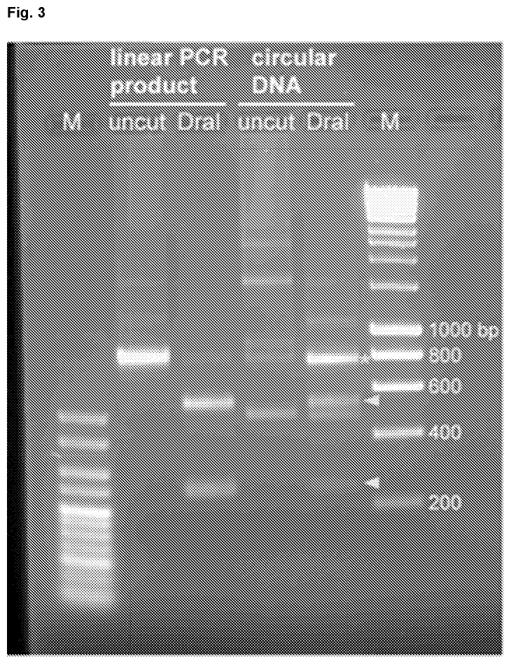

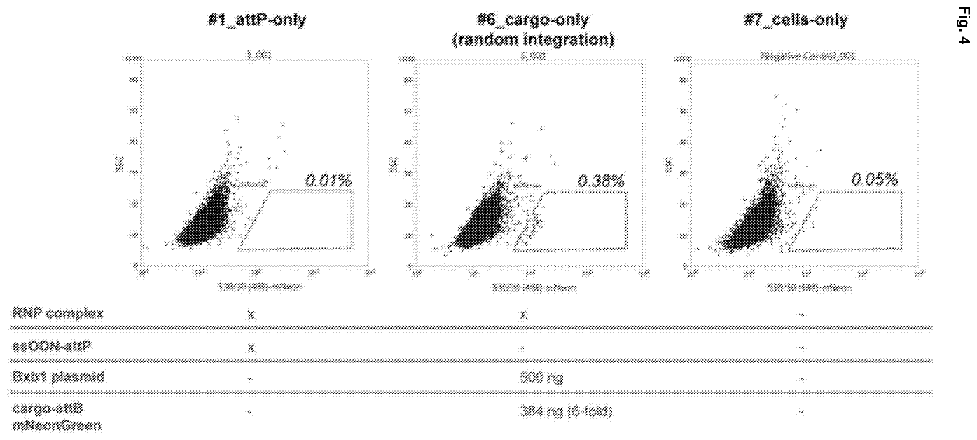

Circularisation efficiency was assessed by restriction digest using an enzyme with a single recognition sequence in the cargo DNA. Digestion of circular DNA resulted in a single band (asterisk), whereas residual linear DNA produced two bands (arrowheads) (FIG. 3).

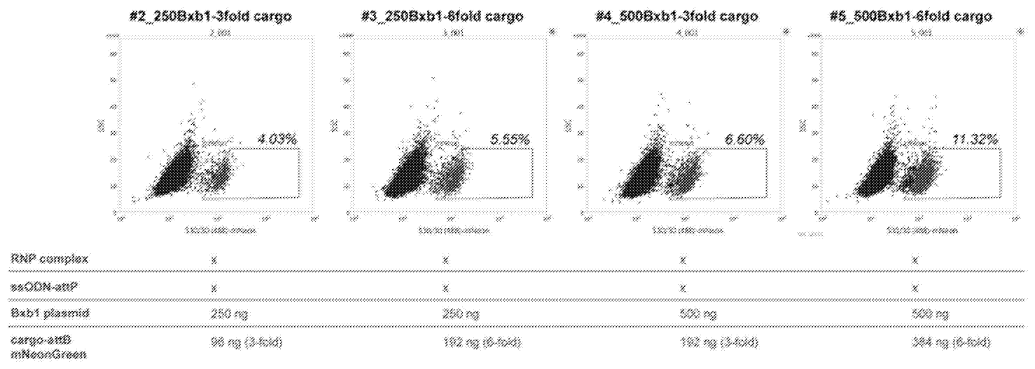

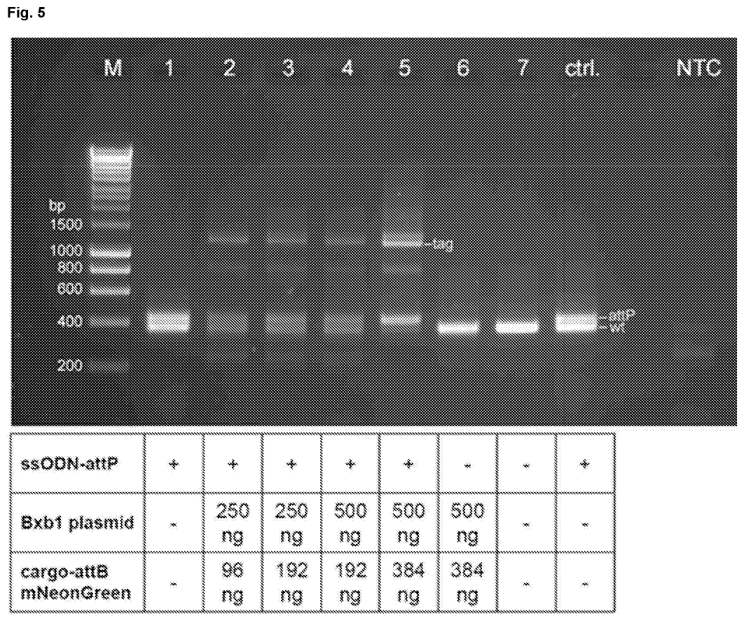

We then delivered the RNP complex, attP ssODN, circularised donor and Bxb1 integrase by nucleofection (Lonza 4D) into 200,000 hIPSCs. We assessed the outcome by flow cytometry analysis of mNeonGreen fluorescence and PCR based genotyping after 7 days (FIGS. 4 and 5). We found that delivery of 24 pmol (4 ug) Cas9, 45 pmol sgRNA, 50 pmol (3.1 ug) of ssODN HDR template, 127 fmol (500 ng) of Bxb1 integrase plasmid and 0.8 pmol (380 ng) of circularised donor gave optimal efficiency. Delivery of all components under these optimised conditions resulted in 11.3% of cells exhibiting mNeonGreen fluorescence (FIG. 4) indicating perfect, in-frame tagging. The levels of background fluorescence were very low (0.01%) when omitting the cargo and Bxb1 plasmids (FIG. 4), and levels of random integration of the tagging plasmid were also low (0.38%) when omitting the ssODN HDR template containing the attP site.

Correct, precise integration was further confirmed by PCR based genotyping (FIG. 5) and Sanger sequencing of the products. This showed that a substantial proportion of alleles had undergone HDR to integrate the attP site, and a large proportion had further undergone recombination to integrate the mNeonGreen cargo, consistent with the observed fluorescence.

### SUMMARY

We demonstrate a new method for one-step integration of transgenes into specific sites within the genome of human iPSCs using a combination of CRISPR/Cas9 targeting and site-specific recombination with the unidirectional serine recombinase, Bxb1. This has a number of advantages over existing methodologies since all components are delivered together in a single nucleofection, it is highly modular and adaptable that avoids the need for cloning HDR template plasmids for each new locus. It is also very precise, and should allow integration of large transgenes of tens of kilobases. These features make it ideal for tagging of endogenous genes with fluorescent proteins, purification tags or regulatable degrons in the context of fundamental research and disease modelling, and also for site-specific integration of large transgenes which is highly beneficial in the context of certain cellular therapeutics such as CAR-T or other synthetic biology applications.

1. A method for gene editing comprising:
delivering simultaneously to a cell;
an endonuclease;
a targeting domain;
a single-stranded oligo DNA nucleotide (ssODN) homology-directed repair (HDR) template comprising a nucleotide sequence encoding a first recombination site;
a DNA recombinase and
a cargo vector comprising a nucleotide sequence encoding a second recombination site and a cargo molecule.

2. The method of claim 1, wherein the endonuclease is delivered as a protein and the DNA recombinase is delivered in an expression vector.

3. The method of claim 1 or 2, wherein the gene editing system is delivered to the cell via electroporation, nucleofection, transfection, lipid-based transfection or nanoparticles.

4. The method of claims 1 to 3, wherein the endonuclease is selected from a Cas9 endonuclease, a Cpf1 (Cas12a) endonuclease, CasX endonuclease, CasY endonuclease, MAD7 endonuclease, transcription activator-like effector nuclease (TALEN), zinc finger nuclease.

5. The method of any one of claims 1 to 4, wherein the targeting domain is selected from a single guide RNA (sgRNA), a zinc finger DNA binding domain, transcription activator-like effector DNA binding domain.

6. The method of any one of claims 1 to 5, wherein the endonuclease is delivered as a ribonucleoprotein (RNP) complex.

7. The method of any of claims 1 to 6, wherein the cargo vector encodes a cargo molecule selected from; a fluorescence marker, a purification tag, a degradation tag, a selectable marker, a gene, a genomic region or cDNA for therapeutics, for differentiation, for metabolic engineering, a genetic locus, guide libraries for CRISPR screening, synthetic DNA fragments, an antibody or fragment thereof, a T-cell receptor, B-cell receptor or chimeric antigen receptor (CAR).

8. The method of claim 7, wherein the degradation tag is selected from an N-degron or a C-degron, preferably wherein the degradation tag is selected from; AID, DHFR, or HALO.

9. The method according to claim 7, wherein the fluorescence marker is selected from GFP, YFP, CFP, BFP, RFP, EGFP, mCherry, mApple, mStrawberry, mOrange, dTomato, tagRFP, tagBFP.

10. The method according to claim 7, wherein the purification tag is selected from Halo, SNAP, TAP, CLIP, FLAG, c-Myc, HA, CBP, or hexa histidine.

11. The method according to claim 7, wherein the selectable marker is selected from puromycin resistance gene, blasticidin resistance gene, neomycin resistance gene, hygromycin resistance gene or delta-TK.

12. The method according to any one of claims 1 to 11, wherein the DNA recombinase is a serine recombinase.

13. The method according to any one of claims 1 to 12, wherein the DNA recombinase is selected from; Bxb1, phiC31, Cre, Flp, KD, B2, B3, R.

14. The method according to any one of claims 1 to 13, wherein the first recombination site is selected from attB, attP, attL, attR, LoxP, FRT.

15. The method according to any one of claims 1 to 14, wherein the second recombination site is selected from attB, attP, attL, attR, LoxP, FRT.

16. The method according to any one of claims 1 to 15, wherein the DNA recombinase is provided as a vector comprising a nucleotide sequence encoding a DNA recombinase, a polypeptide or as an mRNA sequence encoding a DNA recombinase.

17. The method according to any of claims 1 to 16, wherein the method comprises a step of preparing the cargo vector, comprising:
preparing a dsDNA comprising a nucleotide sequence encoding a cargo molecule and a second recombination site, and
circularising the dsDNA to form the cargo vector.

18. The method according to any one of claims 1 to 17, wherein the cargo vector consists of a nucleotide sequence encoding a cargo and a second recombination site.

19. The method according to any one of claims 1 to 18, wherein one or more cargo vectors are delivered to the cell simultaneously.

20. The method according to any one of claims 1 to 19, wherein one or more single-stranded oligo DNA nucleotide (ssODN) homology-directed repair (HDR) template comprising a nucleotide sequence encoding a first recombination site are delivered to the cell simultaneously.

21. The method according to any one of claims 1 to 20, wherein the endonuclease, the targeting domain, the single-stranded oligo DNA nucleotide (ssODN) homology-directed repair (HDR) template comprising a nucleotide sequence encoding a first recombination site, the DNA recombinase and the cargo vector comprising a nucleotide sequence encoding second recombination site and a cargo molecule, are delivered to the cell in vivo, in vitro or ex vivo.

22. The method according to any one of claims 1 to 21, wherein the cell is selected from a mammalian cell or a plant cell, preferably wherein the cell is selected from an iPSC or a pluripotent or somatic stem cell.

23. A system for gene editing comprising:
a Cas9 endonuclease;
a single guide RNA (sgRNA); a single-stranded oligo DNA nucleotide (ssODN) homology-directed repair (HDR) template comprising a nucleotide sequence encoding a first recombination site;
a DNA recombinase and a cargo vector comprising a nucleotide sequence encoding second recombination site; and
a cargo molecule selected from a fluorescence marker, a purification tag, a destabilising tag, or a chimeric antigen receptor (CAR), formulated for simultaneous delivery to a cell.

24. The system for gene editing according to claim 23, wherein the system comprises one or more cargo vector.

25. A kit comprising:
a Cas9 endonuclease;
a single guide RNA (sgRNA);
a single-stranded oligo DNA nucleotide (ssODN) homology-directed repair (HDR) template comprising a nucleotide sequence encoding a first recombination site;
a DNA recombinase and a cargo vector comprising a nucleotide sequence encoding second recombination site; and
a cargo molecule selected from a fluorescence marker, a purification tag, a destabilising tag, or a chimeric antigen receptor (CAR),
formulated for simultaneous delivery to a cell, and optionally instructions for use.

26. A kit according to claim 25, further comprising components which optimise simultaneous delivery of the gene editing system.

27. A composition comprising:
a Cas9 endonuclease;
a single guide RNA (sgRNA);
a single-stranded oligo DNA nucleotide (ssODN) homology-directed repair (HDR) template comprising a nucleotide sequence encoding a first recombination site;
a DNA recombinase;
and a cargo vector comprising a nucleotide sequence encoding second recombination site and a cargo molecule selected from a fluorescence marker, a purification tag, a destabilising tag, or a chimeric antigen receptor (CAR),
formulated for simultaneous delivery to a cell.

28. A method for site specific integration of an exogenous polynucleotide sequence in a cell, comprising
delivering simultaneously to a cell; an endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding second recombination cassette and an exogenous polynucleotide,
maintaining the cell under conditions such that the targeting domain directs endonuclease mediated integration of the first recombination site at a genomic site of interest,
maintaining the cell under conditions such that the DNA recombinase is expressed, contacting the first recombination site and the second recombination site with the DNA recombinase, wherein, the DNA recombinase can mediate site-specific recombination between the first recombination site and the second recombination site to integrate the exogenous polynucleotide into the genomic site of interest.

29. A method of producing a tagged protein comprising;
delivering simultaneously to a cell; an endonuclease, a targeting domain, a single-stranded oligo DNA nucleotide (ssODN) homology-directed repair (HDR) template comprising a nucleotide sequence encoding a first recombination site, a DNA recombinase and a cargo vector comprising a nucleotide sequence encoding a second recombination site and a tag,
maintaining the cell under conditions such that the targeting domain directs endonuclease mediated integration of the first recombination site at a genomic site in proximity to the nucleotide sequence encoding the protein that is to be tagged,
maintaining the cell under conditions such that the DNA recombinase is expressed, allowing the DNA recombinase to contact the first recombination site and the second recombination site, wherein, the DNA recombinase can mediate site-specific recombination between the first recombination site and the second recombination site to integrate the tag into the genomic site of interest, and optionally maintaining the cell under conditions such that expression of a tagged protein can occur.

30. A cargo vector obtained by:
preparing a dsDNA comprising a nucleotide sequence encoding a cargo molecule and a second recombination site, and
circularising the dsDNA to form the cargo vector.
